(19) United States
(12) Patent Application Publication
(10) Pub. No.: US 2020/0261600 A1
(43) Pub. Date: Aug. 20, 2020

(54) METHOD FOR THE TREATMENT OR PREVENTION OF PAIN OR EXCESSIVE NEURONAL ACTIVITY OR EPILEPSY

(71) Applicant: Oxford University Innovation Limited, Oxford (GB)

(72) Inventors: David Laurence Harvey BENNETT, Oxford (GB); John Mansfield DAWES, Oxford (GB); Gregory WEIR, Oxford (GB)

(21) Appl. No.: 16/337,428

(22) PCT Filed: Sep. 28, 2017
(86) PCT No.: PCT/GB2017/052909

§ 371 Date: Mar. 28, 2019

(30) Foreign Application Priority Data
Sep. 29, 2016 (GB) 1616565.6

(51) Int. Cl. A61K 48/00; A61K 38/17; C12N 15/86; C12N 7/00; A61P 25/00

(52) U.S. Cl. CPC A61K 48/0058; A61K 38/1787; A61K 48/0075; C12N 2750/14143; C12N 7/00; A61P 25/00; C12N 15/86

(57) ABSTRACT

The invention relates to methods for the treatment and/or prevention of pain, excessive neuronal activity, or epilepsy, and to gene therapy vectors. In particular, the method comprises the overexpression of a CASPR2 polypeptide in sensory neurons of the individual and the gene therapy vector comprises a polynucleotide sequence that encodes a CASPR2 polypeptide or a variant thereof.

### CROSS-REFERENCE TO RELATED APPLICATIONS

This application is the National Stage of International Application No. PCT/GB2017/052909, filed Sep. 28, 2017, which claims the priority to GB 1616565.6, filed Sep. 29, 2016, which are entirely incorporated herein by reference.

### FIELD OF THE INVENTION

The invention relates to methods for the treatment and/or prevention of pain, excessive neuronal activity, or epilepsy, and to gene therapy vectors.

### BACKGROUND OF THE INVENTION

The treatment of chronic pain is a major unmet clinical need with epidemiological data reporting that 1 in 5 adults are affected despite the use of current analgesics [1]. It has a major impact on an individual's quality of life, limiting their functional status and a more recent study from the United States suggests that it has huge economic costs, much greater than those caused by heart disease, cancer or diabetes [2]. Opioids and NSAIDs are the most common analgesics used. However these rarely provide complete pain relief and are particularly ineffective against chronic pain caused by nerve injury (neuropathic pain) [1]. Furthermore both drug therapies cause severe side-effects [3,4]. Therefore there is a pressing need to identify new pain targets which may help to develop more effective therapies with fewer adverse reactions.

CASPR2 (contactin associated protein 2) is a member of the neurexin superfamily, a group of proteins which function as cell adhesion molecules within the nervous system. Auto-antibodies against this molecule have been linked to a number of clinical syndromes including: neuromyotonia in which there is clinical and electrophysiological evidence of excessive motor unit activity due to enhanced motor axon excitability, Morvan's syndrome in which neuromyotonia is associated with CNS dysfunction such as disrupted sleep and autonomic function and limbic encephalitis characterised by cognitive impairment and epilepsy. A consistent feature described in multiple cohorts of patients seropositive for CASPR2-Ab is the presence of neuropathic pain and indeed in some patients neuropathic pain was the sole presenting symptom [5][6,7]. Furthermore immunosuppression to reduce levels of CASPR2-Ab can lead to a reduction in neuropathic pain [6] suggesting that these antibodies may be pathogenic.

CASPR2 is known to form a protein complex with shaker type potassium channels (STKCs, such as Kv 1.1, 1.2). A reduction in the function of these potassium channels, either through genetic or pharmacological manipulation results in increased DRG neuronal hyperexcitability and increased pain-related behaviour in mice [8-11]. Loss of poatssium channel function in DRG neurons is a key feature of many models of chronic pain, particularly those caused by nerve damage.

### SUMMARY OF THE INVENTION

The inventors have shown for the first time that CASPR2 is directly involved in regulating the excitability of sensory neurons. Mice that do not express CASPR2 (CNTNAP2^{-/-}) have been found by the inventors to have increased pain-related sensitivity. Moreover, they have found that DRG neurons from CNTNAP2^{-/-} mice have fewer kv1.2 membrane channels and have identified a subset of DRG neurons from CNTNAP2^{-/-} mice that have enhanced excitability. Crucially, they have shown that sensory neuron hyperexcitability in CNTNAP2^{-/-} neurons can be rescued by overexpression of CASPR2, suggesting that increased expression of CASPR2 in sensory neurons could be used as a method to treat or prevent pain or excessive neuronal activity or epilepsy.

Accordingly the invention provides a method for the treatment or prevention of pain or excessive neuronal activity or epilepsy in an individual in need thereof, the method comprising overexpression of a CASPR2 polypeptide in sensory neurons of the individual.

The invention also provides
A vector for use in a method of treating or preventing pain, or excessive neuronal activity, or epilepsy in an individual in need thereof, wherein the vector comprises a polynucleotide sequence that encodes a CASPR2 polypeptide or a variant thereof; and
a vector for use in the manufacture of a medicament for treating or preventing pain, excessive neuronal activity or epilepsy in an individual in need thereof, wherein the vector comprises a polynucleotide sequence that encodes a CASPR2 polypeptide or a variant thereof.

The invention also provides a gene therapy vector comprising a polynucleotide sequence that encodes a CASPR2 polypeptide or a variant thereof.

The invention additionally provides a pharmaceutical composition comprising the gene therapy vector.

The invention will now be described in more detail, by way of example and not limitation, and by reference to the accompanying drawings. Many equivalent modifications and variations will be apparent, to those skilled in the art when given this disclosure. Accordingly, the exemplary embodiments of the invention set forth are considered to be illustrative and not limiting. Various changes to the described embodiments may be made without departing from the scope of the invention. All documents cited herein, whether supra or infra, are expressly incorporated by reference in their entirety.

The present invention includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “a vector” includes two or more such vectors.

Section headings are used herein for convenience only and are not to be construed as limiting in any way.

### BRIEF DESCRIPTION OF FIGURES

FIG. 1

CNTNAP2^{-/-} mice display pain-related hypersensitivity. Using Von Frey hairs, CNTNAP2^{-/-} mice had lower paw withdrawal thresholds when compared to wild type littermates indicating hypersensitivity to mechanical stimuli (A). These mice also display hypersensitivity to heat when using the hot plate set at 53° C. (B). In addition CNTNAP2^{-/-} mice display heightened pain-related responses to chemical stimuli. Following the intraplantar application of capsaicin, CNTNAP2^{-/-} showed more nocifensive behavior over a 5 minute period (C,D). Furthermore these mice had an increased formalin response, both in the 1^{st} (0-15 mins) and second phase (15-60 mins).

FIG. 2

DRG neurons from CNTNAP2^{-/-} mice are hyperexcitable. Medium (25-35 μm), but not small (<25 μm), sized neurons from CNTNAP2^{-/-} mice have a reduced threshold for action potential generation (rheobase), when compared to wild type controls in vitro (A). These same neurons fire more action potentials in response to a prolonged suprathreshold stimulus (B). The outward potassium current is also reduced in medium sized DRG neurons from CNTNAP2^{-/-} (D) and an example trace is shown (C). DRG neurons from CNTNAP2^{-/-} have reduced membrane staining of Kv1.2 (E).

FIG. 3

Overexpression of CASPR2 in vitro makes DRG neurons hyposensitive. Kv1.2 membrane expression is lost after 5 days in vitro (DIV), but is still present in the cytoplasm (A). At this time CASPR2 mRNA is also significantly reduced compared to cells cultured for only 1 day (B). Furthermore after 5 DIV, the rheobase is significantly reduced (1 DIV vs eGFP) (C). However overexpression of CASPR2 (CASPR2-egfp) is able to increase the rheobase similar to 1 DIV levels (C). This increase in action potential threshold caused by CASPR2 overexpression can be eliminated with the use of DTX, a Kv channel blocker (C). Similar to the rheobase, the outward potassium current is also reduced following 5 DIV. Again this alteration can be reversed with the overexpression of CASPR2 (D). DTX reverses this effect (D). A representative image of CASPR2-egfp overexpression is shown (E).

FIG. 4

Schematic showing the protein domains of full length CASPR2 and the domains retained in 5 new shorter variants.

FIG. 5

(A) Cell Surface expression of FL-CASPR2 and the 5 new constructs in HEK cells. Constructs were chemically transfected into HEK cells using JetPEI and cells were then incubated for 24 hours. Live cells were treated with WGA conjugated to a red fluorophore for 10 minutes at 37° C. to stain the cell membrane and fixed with 4% PFA. (B) Surface expression of GH. 40× magnification, scale bar is 30 μm.

### DESCRIPTION OF SEQUENCES

SEQ ID NO 1—cDNA sequence CNTNAP2-001 ENST00000361727 (isoform 1)
SEQ ID NO 2—Amino acid sequence CASPR2 (isoform 1)
SEQ ID NO 3—Amino acid sequence Signal peptide—1-27
SEQ ID NO 4—Amino acid sequence Extracellular Domain—28-1262
SEQ ID NO 5—Amino acid sequence Transmembrane Domain—1263-1283
SEQ ID NO 6—Amino acid sequence Cytoplasmic Domain—1284-1331
SEQ ID NO 7—Amino acid sequence extracellular F5/8 type domain—35-181
SEQ ID NO 8—Amino acid sequence extracellular Laminin G-like 1—216-368
SEQ ID NO 9—Amino acid sequence extracellular Laminin G-like 2—401-552
SEQ ID NO 10—Amino acid sequence extracellular EGF-like 1—554-591
SEQ ID NO 11—Amino acid sequence extracellular Fibrinogen C-terminal—592-798
SEQ ID NO 12—Amino acid sequence extracellular Laminin G-like 3—799-963
SEQ ID NO 13—Amino acid sequence extracellular EGF-like 2—963-1002
SEQ ID NO 14—Amino acid sequence extracellular Laminin G-like 4—1055-1214
SEQ ID NO 15—DNA sequence ΔFibC-Lam4
SEQ ID NO 16—Amino acid sequence ΔFibC-Lam4
SEQ ID NO 17—DNA sequence ΔDisc-Lam1
SEQ ID NO 18—Amino acid sequence ΔDisc-Lam1
SEQ ID NO 19—DNA sequence ΔDisc-Lam2
SEQ ID NO 20—Amino acid sequence ΔDisc-Lam2
SEQ ID NO 21—DNA sequence AAV2 ITR
SEQ ID NO 22—DNA sequence AAV2 ITR
SEQ ID NO 23—DNA sequence CAG promoter
SEQ ID NO 24—DNA sequence CASPR2-AB
SEQ ID NO 25—Amino acid sequence CASPR2-AB
SEQ ID NO 26—DNA sequence CASPR2-CD
SEQ ID NO 27—Amino acid sequence CASPR2-CD
SEQ ID NO 28—DNA sequence CASPR2-EF
SEQ ID NO 29—Amino acid sequence CASPR2-EF
SEQ ID NO 30—DNA sequence CASPR2-GH
SEQ ID NO 31—Amino acid sequence CASPR2-GH
SEQ ID NO 32—DNA sequence CASPR2-IJ
SEQ ID NO 33—Amino acid sequence CASPR2-IJ
SEQ ID NO 34—DNA sequence primer F1 Universal
SEQ ID NO 35—DNA sequence primer F2 Universal
SEQ ID NO 36—DNA sequence primer dFibC-lam4 R1 (AB)
SEQ ID NO 37—DNA sequence primer dFibC-lam4 F2 (AB)
SEQ ID NO 38—DNA sequence primer dlam2-EGF1 R1 (CD)
SEQ ID NO 39—DNA sequence primer dlam2-EGF1 F2 (CD)
SEQ ID NO 40—DNA sequence primer dlam1-EGF1 R1 (EF)
SEQ ID NO 41—DNA sequence primer dlam1-EGF1 F2 (EF)
SEQ ID NO 42—DNA sequence primer dlam2-EGF1 R1 (GH)
SEQ ID NO 43—DNA sequence primer dlam2-EGF1 F2 (GH)
SEQ ID NO 44—DNA sequence primer Link dDisc-lam2 R1 (IJ)
SEQ ID NO 44—DNA sequence primer Link dDisc-lam2 F2 (IJ)

### DETAILED DESCRIPTION

### Vector

In some embodiments, the invention relates to vectors, or a gene therapy vectors.

A gene therapy vector is any vector suitable for use in gene therapy, i.e. any vector suitable for the therapeutic delivery of nucleic acid polymers (encoding a CASPR2 polypeptide or a variant thereof) into target cells (sensory neurons) of a patient.

The vector may be of any type, for example it may be a plasmid vector or a minicircle DNA. Typically, the vector is a viral vector. The viral vector may for example be derived from an adeno-associated virus (AAV), a retrovirus, a lentivirus, a herpes simplex virus, or an adenovirus.

### AAV Derived Vectors

The vector may comprise an AAV genome or a derivative thereof.

An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly, the AAV genome of the vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

Typically, the AAV genome of a naturally derived AAV comprises at least one inverted terminal repeat sequence (ITR). An ITR sequence acts in cis to provide a functional origin of replication, and allows for integration and excision of the vector from the genome of a cell. The AAV genome typically also comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV viral particle. Capsid variants are discussed below.

A promoter will be operably linked to each of the packaging genes. Specific examples of such promoters include the p5, p19 and p40 promoters (Laughlin et al., 1979, PNAS, 76:5567-5571). For example, the p5 and p19 promoters are generally used to express the rep gene, while the p40 promoter is generally used to express the cap gene.

The AAV genome may be from any naturally derived serotype or isolate or clade of AAV. Thus, the AAV genome may be the full genome of a naturally occurring AAV virus. As is known to the skilled person, AAV viruses occurring in nature may be classified according to various biological systems.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, identified from primate brain. The serotype of AAV for use in the invention may, for example, be AAV9 Reviews of AAV serotypes may be found in Choi et al (Curr Gene Ther. 2005; 5(3); 299-310) and Wu et al (Molecular Therapy. 2006; 14(3), 316-327).

The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature. The term genetic isolate describes a population of AAV viruses which has undergone limited genetic mixing with other naturally occurring AAV viruses, thereby defining a recognisably distinct population at a genetic level.

Examples of clades and isolates of AAV that may be used in the invention include:

Clade A: AAV1 NC_002077, AF063497, AAV6 NC_001862, Hu. 48 AY530611, Hu 43 AY530606, Hu 44 AY530607, Hu 46 AY530609

Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AY530624, Hu64 AY530625, Hu13 AY530578, Hu56 AY530618, Hu57 AY530619, Hu49 AY530612, Hu58 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, Hu T70 AY695373, Hu T40 AY695372, Hu T32 AY695371, Hu T17 AY695370, Hu LG15 AY695377,

Clade C: Hu9 AY530629, Hu10 AY530576, Hull AY530577, Hu53 AY530615, Hu55 AY530617, Hu54 AY530616, Hu7 AY530628, Hul8 AY530583, Hu15 AY530580, Hu16 AY530581, Hu25 AY530591, Hu60 AY530622, Ch5 AY243021, Hu3 AY530595, Hu1 AY530575, Hu4 AY530602 Hu2, AY530585, Hu61 AY530623

Clade D: Rh62 AY530573, Rh48 AY530561, Rh54 AY530567, Rh55 AY530568, Cy2 AY243020, AAV7 AF513851, Rh35 AY243000, Rh37 AY242998, Rh36 AY242999, Cy6 AY243016, Cy4 AY243018, Cy3 AY243019, Cy5 AY243017, Rh13 AY243013

Clade E: Rh38 AY530558, Hu66 AY530626, Hu42 AY530605, Hu67 AY530627, Hu40 AY530603, Hu41 AY530604, Hu37 AY530600, Rh40 AY530559, Rh2 AY243007, Bb1 AY243023, Bb2 AY243022, Rh10 AY243015, Hu17 AY530582, Hu6 AY530621, Rh25 AY530557, Pi2 AY530554, Pi1 AY530553, Pi3 AY530555, Rh57 AY530569, Rh50 AY530563, Rh49 AY530562, Hu39 AY530601, Rh58 AY530570, Rh61 AY530572, Rh52 AY530565, Rh53 AY530566, Rh51 AY530564, Rh64 AY530574, Rh43 AY530560, AAV8 AF513852, Rh8 AY242997, Rh1 AY530556

Clade F: Hu14 (AAV9) AY530579, Hu31 AY530596, Hu32 AY530597, Clonal Isolate AAVS Y18065, AF085716, AAV 3 NC_001729, AAV 3B NC_001863, AAV4 NC_001829, Rh34 AY243001, Rh33 AY243002, Rh32 AY243003/

The skilled person can select an appropriate serotype, clade, clone or isolate of AAV for use in the present invention on the basis of their common general knowledge.

It should be understood however that the invention also encompasses use of an AAV genome of other serotypes that may not yet have been identified or characterised.

The AAV genome used in the invention may be the full genome of a naturally occurring AAV virus. However, while such a vector may in principle be administered to patients, this will be done rarely in practice. The AAV genome may instead be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid (discussed below) are reviewed in Coura and Nardi (Virology Journal, 2007, 4:99), and in Choi et al. and Wu et al., referenced above.

Derivatives of an AAV genome include any truncated or modified forms of an AAV genome which allow for expression of the CASPR2 polypeptide or variant thereof from the vector in vivo. Typically, it is possible to truncate the AAV genome significantly to include minimal viral sequence yet retain the above function. Reducing the size of the AAV genome in this way allows for increased flexibility in incorporating one or more transgenes and other sequence elements (such as regulatory elements) within the vector. It may also reduce the possibility of integration of the vector into the host cell genome, reduce the risk of recombination of the vector with wild-type virus, and avoid the triggering of a cellular immune response to viral gene proteins in the target cell.

Typically, a derivative will include at least one inverted terminal repeat sequence (ITR), or two ITRs or more. One or more of the ITRs may be derived from AAV genomes having different serotypes, or may be a chimeric or mutant ITR. An example mutant ITR is one having a deletion of a trs (terminal resolution site). This deletion allows for continued replication of the genome to generate a single-stranded genome which contains both coding and complementary sequences i.e. a self-complementary AAV genome. This allows for bypass of DNA replication in the target cell, and so enables accelerated transgene expression.

The one or more ITRs may flank a polynucleotide sequence encoding the CASPR2 polypeptide or variant thereof at either end. The inclusion of one or more ITRs may aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo. The ITR sequences may, for example, be those of AAV2, including those of SEQ ID NOs 21 and 22 and variants thereof.

In some embodiments, ITR elements may be the only sequences retained from the native AAV genome in the derivative. Such a derivative will not include the rep and/or cap genes of the native genome or any other sequences of the native genome.

The following portions could therefore be removed in a derivative of the invention: One inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV virus integrates with a high frequency at a specific site on human chromosome 19, and shows a negligible frequency of random integration, such that retention of an integrative capacity in the vector may be tolerated in a therapeutic setting.

The derivative may be a chimeric, shuffled or capsid modified derivative. Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2, AAV5, AAV6, AAV8 or AAV9. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

### AAV Capsid Coat

A vector comprising an adeno-associated virus (AAV) genome or a derivative thereof will typically have a capsid coat. Such an encapsidated vector may be referred to as an AAV viral particle.

The AAV vectors or particles of the invention include transcapsidated forms wherein an AAV genome or derivative having an ITR of one serotype, for example AAV2, is packaged in the capsid of a different serotype, for example AAV9. The AAV vectors or particles of the invention also include mosaic forms wherein a mixture of unmodified capsid proteins from two or more different serotypes makes up the viral coat. The coat may also comprise modified capsid proteins or variants. The invention encompasses the provision of capsid protein sequences from different serotypes, clades, clones, or isolates of AAV within the same vector or AVV viral particle, i.e. pseudotyping.

The AAV capsid determines the tissue specificity of infection (or tropism) of an AAV virus. Accordingly, the AAV capsid serotypes for use in the invention may be those which have natural tropism for or a high efficiency of infection of the target cells, for example sensory neurons. The capsid serotype may, for example, be AAV2, AAV5, AAV6, AAV8 or AAV9, all of which have successfully targeted mouse DRG. AAV2 and AAV9 have been shown to have a natural tropism for neurons. AAV9 has additionally been shown to be able to cross the BBB to a limited extent. Thus in some embodiments, the capsid serotype may be AAV2 or AAV9. However, one or more of the capsid proteins may be a variant of a capsid protein from the AAV2, AAV5, AAV6, AAV8 or AAV9 serotype or another AAV. For example a variant capsid protein may be chimeric, shuffled, or modified.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type. It may thereby confer improved binding to a target cell or improve targeting or the specificity of targeting of the vector to a particular target cell population, for example, sensory neurons. The unrelated protein or peptide may be a ligand for a component of the blood brain barrier (BBB). In the case of systemically administered AAV vector, a BBB ligand may facilitate crossing of the BBB such that target neurons may be accessed. The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al, referenced above. The AAV vector or particle also includes chemically modified forms bearing ligands adsorbed to the capsid surface. For example, such ligands may include antibodies for targeting a particular cell surface receptor. Relevant sections of the description relating the AAV derived vectors also apply in the case of vectors derived from other sources, such as those discussed further below.

### Retrovirus Derived Vectors

The vector may comprise a retrovirus genome or a derivative thereof.

Derivatives of a retrovirus genome include any truncated or modified forms of a retrovirus genome which allow for expression of the CASPR2 polypeptide or variant thereof from the vector in vivo.

As with AAV derived vectors, a retrovirus derived vector will typically comprise a derivative of a retroviral genome comprising the minimal viral sequences required for packaging and subsequent integration into a host. For retrovirus derived vectors, one or more long terminal repeats (LTRs) are the minimum element required for replication and packaging of the vectors and subsequent integration into the target cell to provide permanent transgene expression. However, other elements may also be present. For example, a human immuno deficiency virus (HIV) derived vector will typically comprises the HIV 5′ LTR, which is necessary for integration into the host cell genome, the Psi signal, which is necessary for packaging of viral RNA into virions, a promoter for the transgene, and the 3′ LTR. Other suitable retroviral vectors may for example be derived from murine leukemia virus (MuLV), gibbon ape leukemia virus (GaLV), Simian Immuno deficiency virus (SIV), and combinations thereof.

The tropism of a retrovirus derived vector is determined by the viral envelope proteins. Targeting of the appropriate cells, for example sensory neurons, may be enhanced by incorporating ligands for the target cells into the viral envelope.

### Adenovirus Derived Vector

The vector may comprise an adenovirus genome or a derivative thereof.

Derivatives of an adenovirus genome include any truncated or modified forms of an adenovirus genome which allow for expression of the CASPR2 polypeptide or variant thereof from the vector in vivo.

A large number of human adenoviral serotypes have been identified and they are categorized into six subgenera (A through F) based on nucleic acid comparisons, fibre protein characteristics, and biological properties. For example, group A includes serotypes 12 and 31, group B includes serotypes 3 and 7, group C includes serotypes 2 and 5, group D includes serotypes 8 and 30, group E includes serotype 4, and group F includes serotypes 40 and 41.

The core of an adenovirus virion contains the linear double-stranded DNA genome and associated proteins V, VII, X (mu), IVa2, and terminal protein (TP). The genome organization of different adenoviruses is conserved and has been proposed to have a timing function, wherein the ends of the genome are transcribed first (the immediate early genes E1 and E4 are located at opposite ends of the linear genome). Early transcription of E1 and E4 leads to the opening of the central region of the genome, allowing transcription of the central region.

Adenoviral genomes typically comprise eight RNA polymerase II transcriptional units: five early units, E1A, E1B, E2A-E2B, E3, and E4; two delayed early units, IX and IVa2; and the Major Late transcriptional unit. The Major Late transcriptional unit is further subdivided into L1-L5 regions based upon the use of alternative splicing sites. The transcriptional units often express proteins of similar function. For example, the E1A unit codes for two proteins responsible for activation of transcription and induction of S-phase upon cellular infection; the E1B transcription unit encodes two proteins that inhibit cellular apoptosis; the E3 transcriptional unit is involved in evasion of the immune response; and the Major Late transcriptional unit encodes structural proteins necessary for assembly of the capsid.

Heterologous transgene sequences may be inserted into the adenoviral genomes, for example in the early transcriptional units and in the coding regions of various structural proteins, such as hexon, penton, and fiber. Deletions may be made in the adenoviral genome (e.g., in the E1 regions) have been used to create replication-defective adenoviral vectors, which have generally been considered safer for administration to human subjects.

In the present invention, the adenovirus may be any adenovirus or derivative suitable for delivery of the transgene to target cells. The adenovirus may be any serotype but is typically Ad5 or Ad2. An adenovirus derived vector of the invention may comprise all or part of the genome of any adenoviral serotype, as well as combinations thereof (i.e., hybrid genomes).

The adenoviral vector used in the invention may be either replication incompetent or replication competent. Such vectors are well known. For example, in a replication incompetent vector the E1 region may be deleted and replaced with an expression cassette with an exogenous promoter driving expression of the heterologous transgene. Usually, the E3 region is also deleted. Deletion of E3 allows for larger inserts into the E1 region. Such vectors may be propagated in appropriate cell lines such as HEK 293 cells which retain and express the E1A and E1B proteins. Other vectors also lack the E4 region, and some vectors further lack the E2 region. E2 and E4 vectors must be grown on cell lines that complement the E1, E4 and E2 deletions.

Vectors may also be helper dependent vectors, which lack most or all of the adenoviral genes but retain cis-acting sequences such as the inverted terminal repeats as well as packaging sequences that are required for the genome to be packaged and replicated. These vectors are propagated in the presence of a helper adenovirus, which must be eliminated from the vector stocks. Once again, such systems are well known in the art.

The capsid is composed of seven structural proteins: II (hexon), III (penton), IIIa, IV (fiber), VI, VII, and IX. The capsid comprises 252 capsomeres, of which 240 are hexon capsomeres and 12 are penton capsomeres. Hexon capsomeres, which are trimers of the hexon protein, make up about 75% of the protein of the capsid. Penton capsomeres, which are pentamers of the penton protein, are situated at each of the 12 vertices of the virion. Each penton capsomer is bound to six adjacent hexon capsomeres and a fiber. The fiber, which is usually a trimer of the fiber protein, projects from the penton capsomer. The hexon protein and, to a lesser extent, the fiber protein comprise the main antigenic determinants of an adenovirus and also determine serotype specificity.

An adenovirus derived vector is particularly suitable for use in the invention when a transient expression of the transgene, ie the polypeptide encoding CASPR2 polypeptide or a variant thereof, is preferred.

### Herpes Simplex Virus Derived Vectors

The vector may comprise an herpes simplex virus (HSV) genome or a derivative thereof.

Derivatives of an HSV genome include any truncated or modified forms of a HSV genome which allow for expression of the CASPR2 polypeptide or variant thereof from the vector in vivo.

Herpes simplex virus (HSV) naturally establishes a life-long latent infection of human peripheral sensory neurons. Recombinant HSV vectors are genetically modified to be incapable of replication, but establish a latent-like state in neurons in vitro and in vivo.

### CASPR2

A CASPR2 polypeptide or variant thereof is any polypeptide that retains trafficking to the membrane of neurons and functions in complex formation with shaker potassium channels such as Kv 1.1 and/or 1.2, and/or regulation of shaker potassium channel function. These activities can be routinely determined by a person skilled in the art. For example, trafficking to cell membranes may be assayed by transfecting HEK cells with a plasmid containing the CASPR2 polypeptide variant incorporating, for the purposes of the assay, a tag such as GFP and visualising membrane expression of the tag/GFP. Interaction and regulation of Kv channels can be assayed by co-expressing plasmids that express Kv channels and the cASPR2 variant in HEK cells or primary DRG cultures and staining for Kv membrane expression. Electrophysiology may be used to measure potassium current and specifically relate this to Kv channels using specific blockers. See also the Examples.

A polynucleotide sequence encoding a variant of CASPR2 is any sequence which encodes such a CASPR2 polypeptide or variant thereof.

Isoform 1 of human CASPR2 (identifier: Q9UHC6-1 [UniParc]) has the amino acid sequence of SEQ ID NO: 2. The first 27 amino acids (SEQ ID NO: 3) are a signal peptide. Amino acids 28-1262 (SEQ ID NO: 4) form the extracellular domain (ECD), amino acids 1263-1283 (SEQ ID NO: 5) form the transmembrane domain, and amino acids 1284-1331 (SEQ ID NO: 6) form the cytoplasmic domain. There is a cytoplasmic 4.1 binding domain that is essential for Kv channel interaction. Therefore Kv channel binding is not expected to be lost by the deletion of portions of the ECD, provided that significant trafficking to the cell membrane is maintained. Accordingly, CASPR2 variants that retain the cytoplasmic domain of SEQ ID NO: 6, the transmembrane domain of SEQ ID NO: 5 and enough of the extracellular domain of SEQ ID NO: 4 to direct trafficking to the cell membrane are expected to be functional CASPR2 variants within the meaning of the invention. However, CASPR2 polypeptides that comprise variants of SEQ ID NO: 6 and/or SEQ ID NO: 5 that retain CASPR2 function as defined above are also encompassed by the invention.

The ECD of isoform 1 has eight annotated domains (http://www.uniprot.org/uniprot/Q9UHC6# section_seq), as shown in Table 1.

TABLE 1
Extracellular domains of CASPR2 isoform 1

| | Amino acids relating | | |
|---|---|---|---|
| Description | SEQ ID NO. 2 | Length | SEQ ID NO. |
| F5/8 type | 35-181 | 147 | 7 |
| (Discoidin) | | | |
| Laminin G-like | 216-368 | 153 | 8 |
| Laminin G-like | 401-552 | 152 | 9 |
| EGF-like 1 | 554-591 | 38 | 10 |
| Fibrinogen C- | 592-798 | 207 | 11 |
| terminal | | | |
| Laminin G-like 3 | 799-963 | 165 | 12 |
| EGF-like 2 | 963-1002 | 40 | 13 |
| Laminin G-like 4 | 1055-1214 | 160 | 14 |

In some cases in accordance with the invention, the polynucleotide sequence encodes a polypeptide having the amino acid sequence of SEQ ID NO: 2 or a variant thereof. The polynucleotide may be a variant of the polynucleotide sequence of SEQ ID NO: 1. A variant of SEQ ID NO: 2 or 1 may comprise further truncations, mutants or homologues of these polypeptides, and any transcript variants thereof which encode a functional CASPR2 polypeptide.

In particular, the variant of SEQ ID NO: 2 or 1 may comprise, or encode for a polypeptide comprising deletions from within the ECD of SEQ ID NO: 4. For example, the variant may comprise a deletion of any one, or any two, any three, any four, any five, any six, or any seven of the domains of Table 1. In some cases the domains that are deleted in the variant are adjacent to each other in full length CASPR2 (SEQ ID NO: 2). In other cases, two, or three, or four, or five, or six of the deleted domains are adjacent to each other in full length CASPR2 (SEQ ID NO: 2). In some embodiments, the variant is one of ΔFibC-Lam4, ΔDisc-Lam1, or ΔDisc-Lam2 as shown in Table 2. In other preferred embodiments, the variant is one of CASPR2-AB, CASPR2-CD, CASPR2-EF, CASPR2-GH and CASPR2-IJ as shown in Table 2. In one embodiment the variant is CASPR2-GH. Each of these variants comprise the deletion of multiple domains from the ECD that are not expected to significantly affect the function of the CASPR2 polypeptide as defined above. Other variants of CASPR2 that have deletions within the EDC, but that retain membrane trafficking, and which are specifically encompassed within the invention, are described in Olsen et al. (2015) and Pinatel et al. (2017). A variant CASPR2 includes polypeptides that comprise further truncations, mutants or homologues of these polypeptides, or of any polypeptide of SEQ ID NO: 16, 18, 20, 25, 27, 29, 31 or 33 and any transcript variants thereof which encode a functional CASPR2 polypeptide, for example polynucleotides of SEQ ID NO: 15, 17 19, 24, 26, 28, 30, 32.

TABLE 2
Preferred CASPR2 polypeptide variants in accordance with the invention.

| | | Nucleotide | Amino acid | Amino acids |
|---|---|---|---|---|
| | | length and | length and | relating |
| CASPR2 type | Domains lost | SEQ ID NO. | SEQ ID NO. | SEQ ID NO. 2 |
| ΔFibC-Lam4 | Fibrinogen-like | 2124 | 707 | 1-591 |
| | Laminin G 3 and 4 | SEQ ID NO. 15 | SEQ ID NO. 16 | 1215-1331 |
| | EGF-like 2 | | | |
| ΔDisc-Lam1 | Discodin | 2997 | 998 | 1-34 |
| | Laminin G 1 | SEQ ID NO. 17 | SEQ ID NO. 18 | 367-1331 |
| ΔDisc-Lam2 | Discodin | 2439 | 812 | 1-34 |
| | Laminin G 1 and 2 | SEQ ID NO. 19 | SEQ ID NO. 20 | 553-1331 |
| CASPR2-AB | Fibrinogen C- | 2127 | 709 | |
| | terminal, | SEQ ID NO: 24 | SEQ ID NO: 25 | |
| | Laminin G-3, | | | |
| | EGF-like 2, | | | |
| | Laminin G-like 4 | | | |
| CASPR2-CD | Laminin G 2, | 3327 | 1109 | |
| | EGF-like 1 | SEQ ID NO: 26 | SEQ ID NO: 27 | |
| CASPR2-EF | Laminin G 1, | 2868 | 956 | |
| | Laminin G 2, | SEQ ID NO: 28 | SEQ ID NO: 29 | |
| | EGF-like 1 | | | |
| CASPR2-GH | Laminin G 2, | 2790 | 930 | |
| | EGF-like 1, | SEQ ID NO: 30 | SEQ ID NO: 31 | |
| | Fibrinogen C- | | | |
| | terminal | | | |
| CASPR2-IJ | Discodin | 2439 | 813 | |
| | Laminin G 1 and 2 | SEQ ID NO: 32 | SEQ ID NO: 33 | |

In certain embodiments, the vectors of the invention have a limited packaging capacity and cannot include the full polynucleotide sequence of full length CASPR2 isoform 1. AAV vectors for example, have a packaging capacity limited to about 4.4 Kb. Using a strong promoter (CAG hybrid ˜1 kb) and by having a bovine ploy A tail sequence (255-bp) for maintaining stability of mRNA, this limits the capacity for transgenes to about 3100 pb. The coding sequence of full length CASPR2 (ENST00000361727.7, SEQ ID NO: 2) is 3996 pb. Accordingly, in certain embodiments, such as those relating to AAV vectors, variant CASPR2 polypeptides that comprise deletions in the ECD that reduce the size of the encoding polynucleotide are preferred. For AAV vectors, a polynucleotide of about 3100 pb or fewer is preferred, for example a polynucleotide encoding ΔFibC-Lam4, ΔDisc-Lam1, ΔDisc-Lam2, CASPR2-AB, CASPR2-EF, CASPR2-GH, CASPR2-IJ or other suitable variant as described in Olsen et al. (2015).

Any homologues mentioned herein are typically at least 70% homologous to a relevant region of SEQ ID NO: 1, 2, 3, 15, 16, 17, 18, 19, 20, 24, 25, 26, 27, 28, 29, 30, 31, 32 or 33.

Homology can be measured using known methods. For example the UWGCG Package provides the BESTFIT program which can be used to calculate homology (for example used on its default settings) (Devereux et al (1984) Nucleic Acids Research 12, 387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/).

In preferred embodiments, a variant sequence may encode a polypeptide which is at least 55%, 65%, 70%, 75%, 80%, 85%, 90% and more preferably at least 95%, 96%, 97%, 98% or 99% homologous to a relevant region or regions of SEQ ID NO: 2, 16, 18, 20, 25, 27, 29, 31 or 33 over at least 50, preferably at least 100, for instance at least 200, 300, 400, 500, 600, 700, 800, 900, 1000, 1100, 1200 or more contiguous amino acids, or even over the entire sequence of the region, regions or variant. The relevant region(s) will be one(s) which provide for functional activity of CASPR2 as defined above. In some cases the relevant regions are each of the domains having the amino acid sequences of SEQ ID NOs: 5 to 14 that are not deleted in the CASPR2 variant relative to full length CASPR2 (SEQ ID NO: 2).

Alternatively, the variant sequence may encode a polypeptide having at least 70%, 75%, 80%, 85%, 90% and more preferably at least 95%, 96%, 97%, 98% or 99% homologous to full-length SEQ ID NO: 2, 16, 18, 20, 25, 27, 29, 31 or 33 over its entire sequence. Typically the variant sequence differs from the relevant region of SEQ ID NO: 2, 16, 18, 20, 25, 27, 29, 31 or 33 by at least, or less than, 2, 5, 10, 20, 40, 50 or 60 mutations (each of which can be substitutions, insertions or deletions).

A variant CASPR2 polypeptide may have a percentage identity with a particular region of SEQ ID NO: 2, 16, 18, 20, 25, 27, 29, 31 or 33 which is the same as any of the specific percentage homology values (i.e. it may have at least 70%, 80% or 90% and more preferably at least 95%, 97% or 99% identity) across any of the lengths of sequence mentioned above.

Variants of SEQ ID NO: 2, 16, 18, 20, 25, 27, 29, 31 or 33 also include truncations. Any truncation may be used so long as the variant is functional, as defined above. Truncations will typically be made to remove sequences that are non-essential for (prenylation) activity and/or do not affect conformation of the folded protein, in particular folding of the active site or relevant binding site. Appropriate truncations can routinely be identified by systematic truncation of sequences of varying length from the N- or C-terminus. Preferred truncations are N-terminal and may remove all other sequences except for the catalytic domain. Preferred truncations may remove all other sequences except for the catalytic, binding, transmembrane and/or intracellular domain and any other sequences needed for proper functioning as set out above.

Variants of SEQ ID NO: 2, 16, 18, 20, 25, 27, 29, 31 or 33 further include mutants which have one or more, for example, 2, 3, 4, 5 to 10, 10 to 20, 20 to 40 or more, amino acid insertions, substitutions or deletions with respect to a particular region of SEQ ID NO: 2, 16, 18, 20, 25, 27, 29, 31 or 33. Deletions, substitutions and insertions are made preferably outside of the cytoplasmic 4.1 binding domain, or outside of the cytoplasmic domain of SEQ ID NO: 6, and/or do not affect conformation of the folded protein.

Substitutions preferably introduce one or more conservative changes, which replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative change may introduce another amino acid that is aromatic or aliphatic in the place of a pre-existing aromatic or aliphatic amino acid. Conservative amino acid changes are well known in the art and may be selected in accordance with the properties of the 20 main amino acids as defined in Table 3 below.

Similarly, preferred variants of the polynucleotide sequence of SEQ ID NO: 1, 15, 17, 19, 24, 26, 28, 30, 32 include polynucleotides having at least 70%, 75%, 80%, 85%, 90% and more preferably at least 95%, 97% or 99% homologous to a relevant region of SEQ ID NO: 1, 15, 17, 19, 24, 26, 28, 30, 32. Preferably the variant displays these levels of homology to full-length SEQ ID NO: 1, 15, 17, 19, 24, 26, 28, 30, 32 over its entire sequence

TABLE 3
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged (+) | Trp | aromatic, hydrophobic, neutral |
| Leu | aliphatic, hydrophobic, neutral | Tyr | aromatic, polar, hydrophobic |

### Promoters and Other Regulatory Elements

In the vector, the nucleic acid encoding the transgene product, i.e. the CASPR2 polypeptide or a variant thereof, is typically operably linked to a promoter. Any suitable promoter may be used as are well known in the art. The promoter may be constitutive i.e. operational in any host cell background. The promoter may be the ubiquitous CAG promoter, which may have the sequence of SEQ ID NO. 23. Alternatively, the promoter may be a cell-specific promoter. A cell-specific promoter is one that drives expression only in, or substantially only in, a particular target cell type. In some embodiments, the target cells are sensory neurons. In other more specific embodiments the target cells are noniceptors or small diameter sensory neurons, or sensory neurons, noniceptors or small diameter sensory neurons at the DRG. Accordingly the promoter may be a nociceptor-specific promoter. A nociceptor-specific promoter is a promoter that selectively expresses the transgene, i.e. the CASPR2 polypeptide or variant thereof, in nociceptors, the neurons that signal pain. For example, the promoter may be a NaV1.8 or peripherin promoter.

One or more other regulatory elements, such as enhancers, postregulatory elements and polyadenylation sites may also be present in addition to the promoter. A regulatory sequence that is operably linked to the transgene is any sequences which facilitate expression of the transgene, for example act to increase expression of a transcript, improve nuclear export of mRNA or enhance its stability. The term “operably linked” refers to a juxtaposition wherein the components described are in a relationship permitting them to function in their intended manner. A control sequence (e.g. a promoter) “operably linked” to a coding sequence (e.g. SEQ ID NO: 1, 15, 17 19, 24, 26, 28, 30 or 32 or a variant thereof) is ligated in such a way that expression of the coding sequence is achieved under conditions compatible with the control sequences.

### Preparation of Vector

The vector(s) of the invention may be prepared by standard means known in the art for provision of vectors for gene therapy. Thus, well established public domain transfection, packaging and purification methods can be used to prepare a suitable vector preparation.

Viral vectors used in gene therapy are typically generated by producing a cell line that packages a nucleic acid vector into a viral particle. The vectors typically contain the minimal viral sequences required for packaging and subsequent integration into a host, as exemplified above, other viral sequences being deleted, leaving capacity for an expression cassette for the polynucleotide(s) to be expressed, i.e. the polynucleotide encoding CASPR2 or a variant thereof. The missing viral functions are typically supplied in trans by the packaging cell line.

Packaging cells are typically used to form virus particles that are capable of infecting a host cell. The packaging cells may be any suitable cell type known in the art. The packaging cells are typically human or human derived cells. Suitable cells include Human Embryonic Kidney (HEK) 293 cells, or HEK 293 derived cell clones (for example to package adenovirus derived vectors), HeLa cells (for example to package HIV or other lentivirus derived vectors) and ψ2 cells or PA317 cells (for example to package retrovirus derived vectors).

As discussed above, AAV derived vectors of the invention may comprise the full genome of a naturally occurring AAV virus in addition to a polynucleotide sequence encoding the CASPR2 polypeptide or variant thereof. However, commonly a derivatised genome will be used, for instance a derivative which has at least one inverted terminal repeat sequence (ITR), but which may lack any AAV genes such as rep or cap.

In such embodiments, in order to provide for assembly of the derivatised genome into an AAV viral particle, additional genetic constructs providing AAV and/or helper virus functions will be provided in a host cell in combination with the derivatised genome. These additional constructs will typically contain genes encoding structural AAV capsid proteins i.e. cap, VP1, VP2, VP3, and genes encoding other functions required for the AAV life cycle, such as rep. The selection of structural capsid proteins provided on the additional construct will determine the serotype of the packaged viral vector.

As mentioned above, AAV viruses are replication incompetent and so helper virus functions, preferably adenovirus helper functions will typically also be provided on one or more additional constructs to allow for AAV replication.

All of the above additional constructs may be provided as plasmids or other episomal elements in the host cell, or alternatively one or more constructs may be integrated into the genome of the host cell.

The invention provides a method for production of a vector of the invention. The method comprises providing a vector which comprises a polynucleotide sequence encoding a CASPR2 polypeptide or a variant thereof in a host cell, and providing means for replication and assembly of said vector into a viral particle. Preferably, the method comprises providing a vector comprising a derivative of an AAV genome and a polynucleotide sequence encoding a CASPR2 polypeptide or a variant thereof, together with one or more additional genetic constructs encoding AAV and/or helper virus functions. Typically, the derivative of an AAV genome comprises at least one ITR. Optionally, the method further comprises a step of purifying the assembled viral particles. Additionally, the method may comprise a step of formulating the viral particles for therapeutic use.

The invention additionally provides a host cell comprising a vector or AAV viral particle of the invention.

### Methods of Therapy and Medical Uses

In some embodiments, the invention comprises a method for the treatment or prevention of pain, or excessive neuronal activity, or epilepsy in an individual in need thereof, the method comprising overexpression of a CASPR2 polypeptide in sensory neurons of the individual. Overexpression of CASPR2 polypeptide may be achieved by any suitable means.

“Overexpression” is relative to expression in the individual before treatment. Hence in an individual who has defective CASPR2 expression or function, “overexpression” of the CASPR2 polypeptide may merely restore CASPR2 levels or function to that in a normal healthy individual, or even sub-normal levels or function of CASPR2.

In some embodiments, the treatment is gene therapy for the treatment or prevention of pain, or excessive neuronal activity, or epilepsy in an individual in need thereof. The term “gene therapy” means the therapeutic delivery of nucleic acid polymers into a patient's cells. In some cases of gene therapy, copies of one or more genes that are normally expressed in a healthy individual are introduced to cells of an individual that has missing or defective copies of the gene or corresponding gene product.

The individual may be a human or a non-human animal. Non-human animals include, but are not limited to, rodents (including mice and rats), and other common laboratory, domestic and agricultural animals, including rabbits, dogs, cats, horses, cows, sheep, goats, pigs, chickens, amphibians, reptiles etc.

The individual may be male or female and any age. In embodiments relating to systemic administration of one or more vectors, the treatment may be more effective in individuals that are seronegative for antibodies to the vector. The antibodies may, for example, be antibodies to an AAV capsid coat. Younger patients are more likely to be seronegative. Accordingly in some cases the individual is preferably paediatric. The individual may preferably up to age 1, 2, 5, 10, 15, 20, 25, or 40.

Many different types of pain can be treated or prevented, as can pain originating from many different sources. Pain may be classified into different types. Nociceptive pain is mediated by pain receptors in response to injury, disease or inflammation. Neuropathic pain is a neurological disorder caused by damage to the pain transmission system from periphery to brain. Examples are diabetic neuropathy, trigeminal neuralgia, HIV-evoked neuropathy or antiviral neuropathy. Psychogenic pain is pain associated with actual mental disorder.

Pain may be chronic or acute, depending on its duration. Chronic pain can generally be described as pain that has lasted for a long time, for example beyond the expected period of healing. Typically, chronic pain is pain which lasts for 3 months or more. Pain which lasts for less than 30 days can be classed as acute pain, and pain of intermediate duration can be described as moderate or subacute pain.

The present invention relates to the treatment of any type of pain. The pain treated by the present invention may be associated with, for example, symptoms associated with one or more of inflammation (for example from cancer, arthritis or trauma), back pain (including sciatic back pain), trapped nerve, arthritic pain, cancer-related pain, dental pain, endometriosis, birthing-related pain (e.g. pre- and/or post-partum), post-surgical pain or trauma. In particular, the moderate to chronic pain may be associated with inflammation, back pain, arthritis or cancer-related pain, particularly inflammation or cancer-related pain. The pain treated in relation to the present invention is typically chronic and/or neuropathic pain. The pain is typically mediated by nociceptive and/or neuropathic mechanisms.

In some embodiments, the method is for the treatment of any disease or condition associated with CASPR2 autoantibodies, or with a loss of function of CASPR2, or with excessive neuron activity, including neuromyotonia, Morvan's syndrome, limbic encephalitis or epilepsy, including recessive symptomatic focal epilepsy.

### Pharmaceutical Compositions and Modes of Administration

The one or more vectors, or the gene therapy vector, of the invention can be formulated into pharmaceutical compositions. These compositions may comprise, in addition to the vector(s), a pharmaceutically acceptable excipient, carrier, buffer, stabiliser or other materials well known to those skilled in the art. Such materials should be non-toxic and should not interfere with the efficacy of the active ingredient. Examples of suitable compositions and methods of administration are provided in Esseku and Adeyeye (2011) and Van den Mooter G. (2006). The precise nature of the carrier or other material may be determined by the skilled person according to the route of administration. Examples of techniques and protocols can be found in Remington's Pharmaceutical Sciences, 20th Edition, 2000, pub. Lippincott, Williams & Wilkins.

The vectors of the invention may be administered by any suitable route and means that allows for transduction of the target cells. The target cells are neurons. More specifically, the target cells may be sensory neurons or motor neurons. The sensory neurons may more specifically be small diameter sensory neurons (which are enriched for noniceptors) or noniceptors, or cells of the the sensory ganglia. The target may more specifically be sensory neurons, small diameter sensory neurons, noniceptors or motor neurons at the dorsal root ganglion.

In order to reach the CNS, or the DRG, a vector must cross or bypass the blood brain barrier (BBB). Intrathecal administration, which is commonly used for certain analgesics, is by injection into the spinal canal or into the subarachnoid space. Accordingly, the vectors of the invention may be administered intrathecally. Other routes of administration that bypass the BBB to allow transduction of the CNS or the DRG that could be used include intrathecal, intraneural (for example into the sciatic nerve), intra-DRG and lumbar puncture.

In some embodiments the vectors may be administered systemically, for example by intravenous administration. In contrast to other AAV, AAV9 delivered systemically has been shown to cross the BBB to a limited extent (Forsayeth and Bankiewicz, 2011). The invention also encompasses viral vectors, for example AAV-derived vectors, comprising capsids that are engineered to target BBB ligands. The BBB ligands facilitate transduction the vector across the BBB.

Dosages and dosage regimes can be determined within the normal skill of the medical practitioner responsible for administration of the composition. The dose of a vector of the invention may be determined according to various parameters, especially according to the age, weight and condition of the patient to be treated; the route of administration; and the required regimen. A physician will be able to determine the required route of administration and dosage for any particular patient.

Administration is typically in a “prophylactically effective amount” or a “therapeutically effective amount” (as the case may be, although prophylaxis may be considered therapy), this being sufficient to show benefit to the individual. For example, a prophylactically effective amount or therapeutically effective amount of the vectors of the invention may be an amount that is sufficient to result in expression of the CASPR2 polypeptide, or variant thereof, in the target sensory neurons of the individual, and/or to reduce the perception of pain, excessive neuronal activity, or the symptoms or epilepsy.

A typical single dose of the one or more vectors of the invention is between 10⁹ and 10¹⁵, or 10¹⁰ and 10¹⁴, or 10¹¹ and 10¹² viral genomes (vg). A dose at the lower end of the range will typically be used for administration direct to the CNS, whilst a dose at the higher end of the range will typically be needed for systemic administration. A single AAV capsid that contains a single stranded DNA molecule is a single viral genome (vg). Vg can be quantified by any suitable method as well known in the art, for example real-time PCR. The one or more vectors are preferably administered only once, resulting, depending on the vector used, in permanent or transient expression of the CASPR2 polypeptide, or variant thereof, in target sensory neurons of the individual, but repeat administrations, for example in future years and/or with different serotypes may be considered.

A composition of the invention may be administered alone or in combination with other therapeutic compositions or treatments.

### EXAMPLES

### Example 1

### CASPR2 Regulates Pain-Related Hypersensitivity in Mice

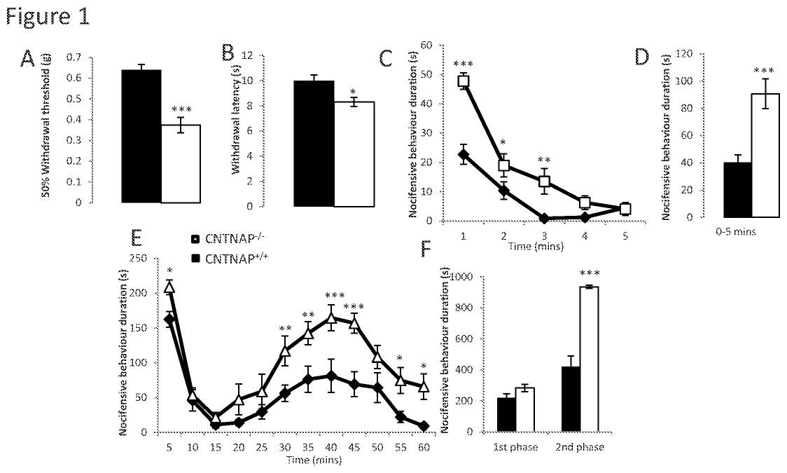

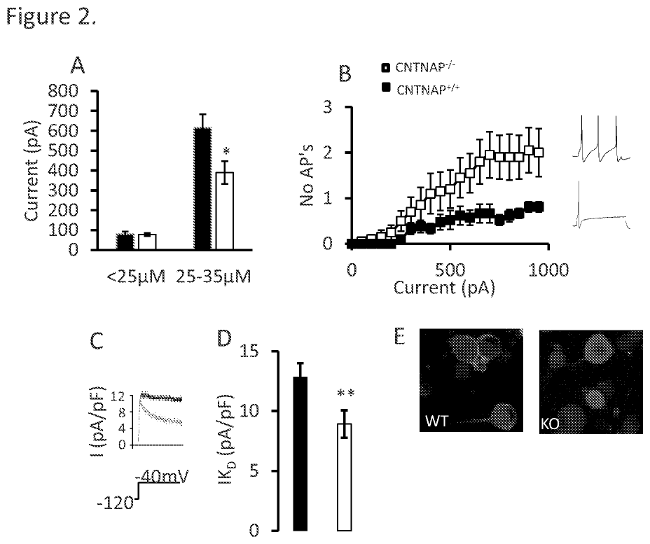

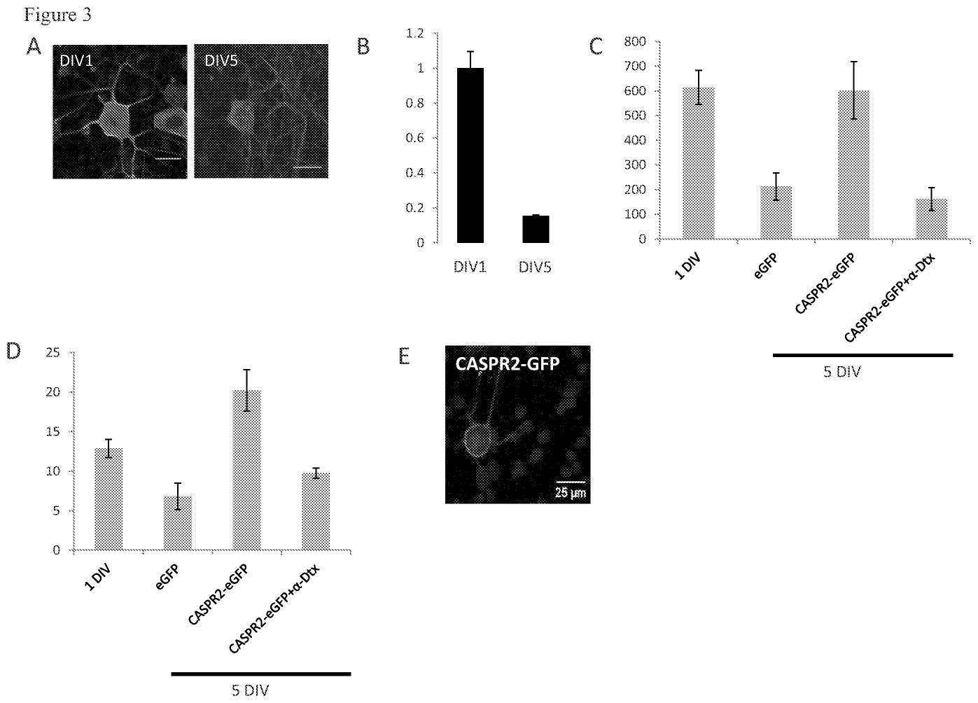

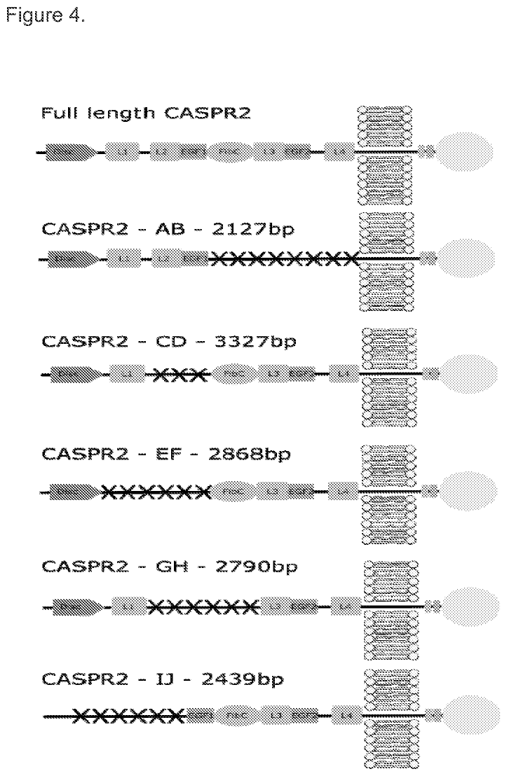

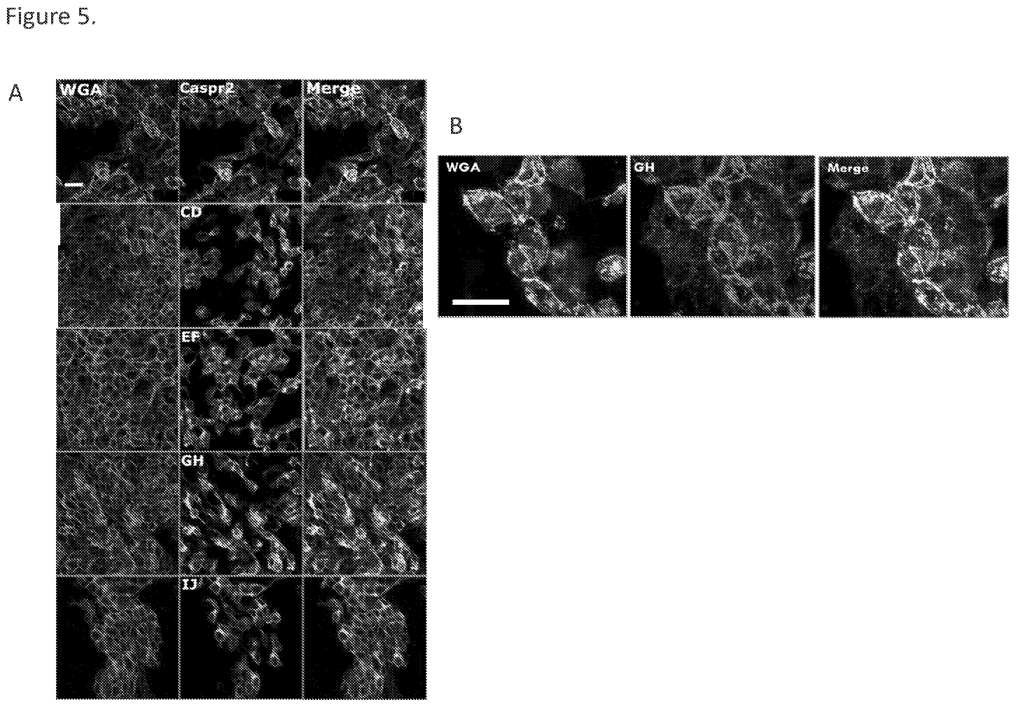

We hypothesised that CASPR2 expressed in DRG neurons may be important in regulating pain-related hypersensitivity through its regulation of STKC function in DRG neurons. We therefore investigated whether a loss of CASPR2 might alter pain-related behaviour in mice. Using mice which no longer express CASPR2 (termed CNTNAP ^{-/-}) [12], we found that a loss of CASPR2 resulted in pain-related hypersensitivity (FIG. 1). Using Von Frey hairs, CNTNAP ^{-/-} mice had a significantly lower withdrawal threshold than wild type littermates (CNTNAP ^{+/+}0.64±0.06 g vs CNTNAP ^{-/-}0.37±0.04 g) meaning these mice are hypersensitive to mechanical stimuli (FIG. 1A). We also assessed sensitivity to thermal stimuli (FIG. 1B). Using a hot plate set at 53° C. there was a significant difference between groups with the CNTNAP ^{-/-} mice having a shorter latency to response (CNTNAP ^{+/+}10.0±0.05 vs CNTNAP ^{-/-}8.3±0.4, FIG. 1B) suggesting that a loss of CASPR2 results in hypersensitivity to supra-threshold noxious heat stimuli. Sensitivity to chemical algogens was also assessed. In CNTNAP ^{+/+} the intraplantar injection of capsaicin (a TRPV1 agonist) resulted in overt pain behaviour which was particularly marked within the first minute and then largely absent after the third (FIG. 2C). In CNTNAP ^{-/-} mice the same intraplantar injection of capsaicin produced a significantly augmented pain response and the duration of this response was increased (FIG. 1C&D). Loss of CASPR2 also resulted in an enhanced response to the formalin test both in the first (0-15 mins) and second phase (15-60 mins) post injection (FIG. 1E & F). By measuring the duration of biting/licking/lifting of the paw, overt pain behaviour was measured. The initial response (0-5 mins) in CNTNAP ^{-/-} was significantly greater when compared to mice expressing CASPR2 (CNTNAP ^{+/+}162.5±11.3 vs CNTNAP ^{-/-}208.5±10.5). This difference subsided, but was evident again in the second phase (FIG. 1E & F) and while pain behaviour in littermate controls mice was gone after 1 hour, it was still present in mice lacking CASPR2. These results therefore show that a lack of CASPR2 protein in mice leads to increased pain-related sensitivity.

### Example 2

### CASPR2 Regulates Sensory Neuronal Excitability and Membrane Kv Channel Expression

We next used patch clamp analysis to assess DRG cell excitability in these mice. We found that medium sized DRG neurons (25-35 μm) from CNTNAP ^{-/-} mice had a significantly lower rheobase (current threshold for action potential) than controls (CNTNAP ^{+/+}614.5±69.4 pA vs CNTNAP ^{-/-}389.2±57.6 pA, FIG. 2A) consistent with enhanced excitability. This was not true of small neurons (>25 μm) consistent with the expression profile of CASPR2 within these cells. These medium sized neurons from CNTNAP ^{-/-} mice also generated significantly more action potentials in response to suprathreshold stimuli (FIG. 2B). We hypothesised that this hyperexcitability phenotype was due to a loss of functional Kv1 channels in these neurons and therefore assessed I_{KD}, a slowly inactivating voltage-dependent K⁺ current that is formed by kv1.1 and 1.2 channels [13]. Since I_{KD} is active at subthreshold membrane potentials we measured outward current in response to a membrane potential change from −120 mV to −40 mV. In DRG neurons from control mice this change in membrane potential produced a clear outward current (FIG. 2C). However, in CNTNAP ^{-/-} DRG neurons the current density was reduced particularly over time (FIG. 2C) and this was significantly different between genotypes (CNTNAP ^{+/+}12.9±12.9 pA/pF vs CNTNAP ^{-/-}8.9 pA/pF, FIG. 2D). One possibility for this decreased Kv1 current is that CASPR2 is important for their trafficking to the cell membrane, something which it has been shown to do for other ion channels [14]. We therefore assessed membrane staining of Kv1.2 in DRG neurons from CNTNAP ^{+/+} and CNTNAP ^{-/-} mice and indeed found a decrease in kv1.2 membrane expression in mice lacking CASPR2 (FIG. 2 E). These results indicate that CASPR2 is an important molecule in regulating the excitability of sensory neurons.

### Example 3

Since a loss of CASPR2 leads to hyper-excitability, as shown in Examples 2 and 3, we wanted to assess whether the reverse was true by overexpressing CASPR2 in vitro. In cultured DRG neurons from wild type mice at 1 day in vitro (DIV), NF200 positive cells of 25-35 μm in diameter show membrane staining of Kv1.2 (FIG. 3A). However after 5 DIV this membrane expression is lost (FIG. 3A), which coincides with a reduction in CASPR2 expression levels (FIG. 3B). Consistent with these changes, cells become hyperexcitable and show a reduced I_{KD} (FIG. 3D) comparing 1 DIV to the control GFP expressing neurons at 5 DIV. We therefore set out to rescue this phenotype by overexpressing CASPR2. DRG cells were electroporated with plasmids containing either CASPR2 tagged by eGFP in the cytoplasmic domain or a plasmid containing eGFP only (control). Overexpression of CASPR2-eGFP resulted in membranous eGFP staining in a subset of DRG neurons (FIG. 3 E). At 5 DIV, CASPR2 overexpressing cells had a significantly higher rheobase than eGFP expressing cells (eGFP 213.7±55.5 pA vs CASPR2 602.0±115.9 pA, FIG. 3 C). The rheobase in the CASPR2 group was similar to that seen after 1 DIV in control DRG cells (1 DIV 614.5±69.4 pA, FIG. 3 C). Interestingly this effect was lost with DTX (a toxin that blocks STKCs) treatment suggesting that the reduced excitability seen with CASPR2 overexpression was due to enhanced activity of Kv1 channels (FIG. 3 C). In agreement I_{KD} was also greater in the CASPR2-eGFP vs eGFP control group (FIG. 3D).

### Interpretation of Data

This data shows for the first time that CASPR2 is an important molecule in regulating sensory neuronal excitability and pain-related behaviour in mice. The likelihood that CASPR2 also has a similar role in humans is enhanced since patients who make autoantibodies against this protein develop chronic neuropathic pain. A loss of potassium function and DRG neuronal hyperexcitability are hallmarks of chronic pain models, particularly those caused by nerve injury. Here we have used an in vitro model, where after 5 days in culture cells become hyperexcitable due to a loss of membrane Kv1 expression. Overexpression of CASPR2 was able to rescue this phenotype back to control levels. This data suggests that the overexpression of CASPR2 is a viable option for reducing DRG neuronal hyper-excitability through its capacity to increase the functional expression of STKCs.

Severe side-effect due to actions within the CNS can be a major limiting factor for many therapies. For example opioids can provide extremely effective pain relief. However, they also cause sedation, physical dependence and addiction. Therefore by designing a therapy that specifically targets primary sensory neurons which are located outside of the CNS, this approach should limit such adverse events.

### Methods

### Mice

All procedures were carried out in accordance with UK home office regulations and in line with the Animals Scientific procedures Act 1986 at a licenced facility within the University of Oxford. Animals were housed in temperature and humidity controlled rooms where food and water was available ad libitum, with a 12 hour light dark cycle. CNTNAP2^{-/-} mice, on a C57Bl/6 background, were obtained from The Jackson Laboratory. Their generation has previously been described [12]. Heterozygous mice were bred together to obtain both CNTNAP2^{-/-} mice and littermate controls (CNTNAP2^{+/+}). Both male and female adult mice were used for experimental studies.

### Behavioural Studies

For all behaviour testing, mice were acclimatised to the testing equipment and baseline values were obtained by averaging data from 2-3 sessions. Mechanical sensitivity was assessed using calibrated Von Frey hairs. These were applied to the plantar surface of the hind paw and a reflex withdrawal response was used to calculate the 50% withdrawal threshold [15]. Response to a suprathreshold heat stimulus was measured using the hot plate assay. A metallic plate was set so that the surface temperature was 53° C. Mice were then placed onto the plate and the latency until a response, in this case shaking, licking or biting of the paw, was measured. For analysis of sensitivity to caspsaicin, 1.5 ug of capsaicin diluted in 10 ul of sterile saline (1% ethanol, 0.5% Tween) was injected into one hindpaw and the animals were immediately placed into a Perspex cylinder. Over a 5 minute period the duration of over pain behaviour (biting/licking/lifting) was measured and recorded at for each minute. For the formalin test, 20 ul of 5% formalin diluted in sterline saline was injected into one hindpaw and the duration of overt pain behaviour (biting/licking/lifting) was measured over a 1 hour period.

### DRG Neuronal Culture

Mice were culled using a CO2 chamber and DRG extracted following a laminectomy. DRG were then placed in collagenase for 1-1.5 hours at 37° C. Following enzymatic digestion, DRG neurons were mechanically dissociated and placed onto laminin treated coverslips in culture medium supplemented with growth factors (NGF and GDNF) for 24-48 hours. For overexpression studies, cells were electroporated with plasmids containing either CASPR2-egfp or egfp alone, driven by the CMV promoter, before plating and cultured for 5 days to allow for gene expression.

### Immunohistochemistry

DRG cells were fixed using 4% PFA and washed with PBS. Coverslips were then incubated with primary antibody overnight at 4° C. in PBS-TritonX and washed with PBS. Secondary antibodies were then placed onto coverslips for 2 hours at romm temperature and slides were again washed before mounting onto slides. Imaging was performed with a Zeiss Confocal microcope.

### Electrophysiology

Cultured DRG neurons were tested after 24-48 hours for CNTNAP2^{-/-} vs CNTNAP2^{+/+} experiments, or after 5 days for overexpression studies. Whole cell patch clamp analysis was used to determine the rheobase and outward current.

### Example 4

We designed new variants of CASPR2 of reduced size and tested for whether the variants maintained their membrane trafficking ability._Five constructs were generated from a plasmid containing cDNA encoding the full length version of human CASPR2 using In-Fusion cloning (Takara Bio, Clontech). This approach allows for enzymatic cloning of separate sequences into the same vector. To this purpose, we designed primers for each insert to include a 15 bp of overlapping sequence with the linearised host vector and the adjacent insert. Then, complimentary and overlapping sequences were joined together in the cloning reaction. We designed 4 primers per construct as described by manufacturer. As all 5 constructs had identical starting and ending sequences and were cloned into the same pEGFP-N1 vector, the first and the last primers, guiding the inserts into the vector, were universal and could be applied for all 5 constructs. In addition, the design of the primers aimed to retain the XhoI and XmaI restriction sites on each end of the construct. Also, at its C-terminal, each of the 5 constructs was fused to eGFP, to allow for easy analysis of its cellular localisation. The primer sequences are shown in Table 4. Schematics of full length CASPR2 and each new CASPR2 variant are shown in FIG. 4.

TABLE 4
Primer sequences used for new CASPR2 construct design

| | |
|---|---|
| CTACCGGACTCAGATCTCGAGATGCAGGCGGCECCGCGC | F1 Universal |
| TGGCGACCGGTGGATCCCGGGAAATGAGCCATTCCTTTTGC | R2 Universal |
| GCGAGGCCCCGTGGCAGGTGGCCCCACTGTATCC | dFibC-lam4 R1 (AB) |
| CACCTGCCACGGGGCCTCGCCGCTGACC | dFibC-lam4 F2 (AB) |
| AGATAGAGTTACAAGAAAAGCTCAAATTTCCCAC | dlam2-EGF1 R1 (CD) |
| CTTTTCTTGTAACTCTATCTACGAGCCTCC | dlam2-EGF1 F2 (CD) |
| AGATAGAGTTGTTCAAGGCAATGACATCTTTCAG | dlam1-EGF1 R1 (EF) |
| TGCCTTGAACAACTCTATCTACGAGCCTTCC | dlaml-EGF1 F2 (EF) |
| CTCCAACCACACAAGAAAAGCTCAAATTTCCCAC | dlam2-FibC R1 (GH) |
| CTTTTCTTGTGTGGTTGGAGATACTGACCG | dlam2-FibC F2 (GH) |
| TGTCTATGATTTTTTGGGACGTGGAGGGAGC | Link dDisc-lam2 R1 (IJ) |
| GTCCCAAAAAATCATAGACAGATGTGTGCCC | Link dDisc-lam2 F2 (IJ) |

Our focus was to generate a multidomain deletion constructs small enough to fit into an AAV vector containing a strong promoter while also retaining the function of Caspr2 to traffic components of voltage gated potassium channel-complexes (VGKCs)/STKCs (e.g. Kv1.1 and Kv1.2) to the cell surface. AB and IJ were designed based on data from Olsen et al. (2015) (PMID: 26185774). Olsen et al. (2015) shows that when expressed in HEK cells these variants were able to reach the cell membrane and were of suitable size.

Caspr2 is a part of the VGKC complex. Interaction between Caspr2 and Tag1 (also known as contactin-2) is thought to be important for the function of the complex and for Caspr2 cell surface expression (Traka et al. (2003)(PMID: 12975355). Therefore, we chose construct CD as it was shown to retain the ability of full length Caspr2 to interact with Tag1 (Pinatel et al. (2017)(PMID 28533267). Construct CD is 3.3 kb long, which exceeds the limit of 3.1 kb for inclusion in an AAV vector. Therefore, we designed constructs EF and GH by deleting one additional domain on the left or the right side of the deletion in construct CD.

We tested each variant for cell surface expression in HEK cells. The cell membrane was marked using WGA (left hand column, FIG. 5A) and CASPR2 (full length or variant) localisation was shown by GFP expression (middle column, FIG. 5A). Clear membrane staining can be seen for FL-CASPR2 in HEK cells, indicated by the presence of bright staining in the merged image (right hand column, FIG. 5A). This full length version of CASPR2 produces clear reduction of hyperexcitability in DRG neurons, as described above. All constructs presented here show cell surface expression and therefore may be expected to have a similar action in DRG neurons as the full length version (FIG. 5A). CASPR2-GH produced the most clear cell surface expression of the five new constructs and a higher power image is shown in FIG. 5B.

### REFERENCES

1. Breivik H, Collett B, Ventafridda V, Cohen R, Gallacher D: Survey of chronic pain in Europe: Prevalence, impact on daily life, and treatment [Internet]. Eur. J. Pain 2006, 10:287-333.

2. Gaskin D J, Richard P: The economic costs of pain in the United States. J. Pain 2012, 13:715-724.

3. Ng SC, Chan F K L: NSAID-induced gastrointestinal and cardiovascular injury. Curr. Opin. Gastroenterol. 2010, 26:611-617.

4. R K RALH: Opioids and the treatment of chronic pain: controversies, current status, and future directions. Exp Clin Psychopharmacol 2008, 16:405-416.

5. Irani S R, Pettingill P, Kleopa K A, Schiza N, Waters P, Mazia C, Zuliani L, Watanabe O, Lang B, Buckley C, et al.: Morvan syndrome: Clinical and serological observations in 29 cases. Ann. Neurol. 2012, 72:241-255.

6. Klein C J, Lennon V A, Aston P A, McKeon A, Pittock S J: Chronic pain as a manifestation of potassium channel-complex autoimmunity. Neurology 2012, 79:1136-1144.

7. Lancaster E, Huijbers M G M, Bar V, Boronat A, Wong A, Martinez-Hernandez E, Wilson C, Jacobs D, Lai M, Walker R W, et al.: Investigations of caspr2, an autoantigen of encephalitis and neuromyotonia. Ann. Neurol. 2011, 69:303-311.

8. Everill B, Rizzo M a, Kocsis J D: Morphologically identified cutaneous afferent DRG neurons express three different potassium currents in varying proportions. J. Neurophysiol. 1998, 79:1814-1824.

9. Gold M S, Shuster M J, Levine J D: Characterization of six voltage-gated K+ currents in adult rat sensory neurons. J. Neurophysiol. 1996, 75:2629-2646.

10. Hao J, Padilla F, Dandonneau M, Lavebratt C, Lesage F, Noël J, Delmas P: Kv1.1 channels act as mechanical brake in the senses of touch and pain. [Internet]. Neuron 2013, 77:899-914.

11. Zhao X, Tang Z, Zhang H, Atianjoh F E, Zhao J-Y, Liang L, Wang W, Guan X, Kao S-C, Tiwari V, et al.: A long noncoding RNA contributes to neuropathic pain by silencing Kcna2 in primary afferent neurons. [Internet]. Nat. Neurosci. 2013, 16:1024-31.

12. Poliak S, Salomon D, Elhanany H, Sabanay H, Kiernan B, Pevny L, Stewart C L, Xu X, Chiu S Y, Shrager P, et al.: Juxtaparanodal clustering of Shaker-like K+ channels in myelinated axons depends on Caspr2 and TAG-1. J. Cell Biol. 2003, 162:1149-1160.

13. Madrid R, de la Peña E, Donovan-Rodriguez T, Belmonte C, Viana F: Variable threshold of trigeminal cold-thermosensitive neurons is determined by a balance between TRPM8 and Kv1 potassium channels. J. Neurosci. 2009, 29:3120-3131.

14. Varea O, Martin-de-Saavedra M D, Kopeikina K J, Schürmann B, Fleming H J, Fawcett-Patel J M, Bach A, Jang S, Peles E, Kim E, et al.: Synaptic abnormalities and cytoplasmic glutamate receptor aggregates in contactin associated protein-like 2/Caspr2 knockout neurons. [Internet]. Proc. Natl. Acad. Sci. U. S. A. 2015, 112:6176-81.

15. Chaplan S R, Bach F W, Pogrel J W, Chung J M, Yaksh T L: Quantitative assessment of tactile allodynia in the rat paw. J. Neurosci. Methods 1994, 53:55-63.

16. Olsen A L, Lai Y, Dalmau J, Scherer S S, Lancaster E: Caspr2 autoantibodies target multiple epitopes. Neurol Neuroimmunol Neuroinflamm. 2015, 2(4):e127

17. Traka M, Goutebroze L, Denisenko N, Bessa M, Nifli A, Havaki S, Iwakura Y, Fukamauchi F, Watanabe K, Soliven B, Girault J A, Karagogeos D.: Association of TAG-1 with Caspr2 is essential for the molecular organization of juxtaparanodal regions of myelinated fibers. J Cell Biol. 2003 September 15;162(6):1161-72.

18. Pinatel D, Hivert B, Saint-Martin M, Noraz N, Savvaki M, Karagogeos D, Faivre-Sarrailh C.: The Kv1-associated molecules TAG-1 and Caspr2 are selectively targeted to the axon initial segment in hippocampal neurons. J Cell Sci. 2017 July 1;130(13):2209-2220.

1. A method for the treatment or prevention of pain, or excessive neuronal activity, or epilepsy in an individual in need thereof, the method comprising overexpression of a CASPR2 polypeptide in sensory neurons of the individual.

2. The method according to claim 1, comprising administering a vector that comprises a polynucleotide sequence that encodes a CASPR2 polypeptide or a variant thereof.

3. The method of claim 1, wherein the vector is derived from a viral vector selected from the group consisting of an adeno-associated virus (AAV) vector, a lentiviral vector, a herpes simplex virus vector, a retroviral vector and an adenoviral vector.

4. The method of claim 1, wherein the vector comprises an adeno-associated virus (AAV) genome or a derivative thereof.

5. The method of claim 4, wherein the vector has a capsid coat of serotype AAV2, AAV5, AAV6, AAV8 or AAV9.

6. The method of claim 1, wherein the vector is administered intrathecally.

7. A gene therapy vector comprising a polynucleotide sequence that encodes a CASPR2 polypeptide or a variant thereof.

8. The gene therapy vector of claim 7, wherein the vector is derived from a viral vector selected from the group consisting of an adeno-associated virus (AAV) vector, a lentiviral vector, a herpes simplex virus vector, a retroviral vector and an adenoviral vector.

9. The gene therapy vector of claim 7, comprising an adeno-associated virus (AAV) genome or a derivative thereof.

10. The gene therapy vector of claim 9, having a capsid coat of serotype AAV2, AAV5, AAV6, AAV8 or AAV9.

11. The method of claim 4, wherein the capsid serotype is AAV9.

12. The method of claim 1, wherein the CASPR2 polypeptide comprises the amino acid sequence of SEQ ID NO: 2, 25, 27, 29, 31 or 33 or a variant thereof.

13. A pharmaceutical composition comprising the gene therapy vector of claim 7.

14. A vector for use in a method of treating or preventing pain, or excessive neuronal activity, or epilepsy in an individual in need thereof, wherein the vector comprises a polynucleotide sequence that encodes a CASPR2 polypeptide or a variant thereof.

15. The method of claim 1, wherein the pain is neuropathic pain.

16. The method of claim 1, wherein the excessive neuronal activity is associated with neuromyotonia, Morvan's syndrome, limbic encephalitis or epilepsy.

17. The gene therapy vector of claim 7, wherein the capsid serotype is AAV9.

18. The gene therapy vector of claim 7, wherein the CASPR2 polypeptide comprises the amino acid sequence of SEQ ID NO: 2, 25, 27, 29, 31 or 33 or a variant thereof.

19. The vector for use of claim 14, wherein the pain is neuropathic pain.

20. The vector for use of claim 14, wherein the excessive neuronal activity is associated with neuromyotonia, Morvan's syndrome, limbic encephalitis or epilepsy.


SEQUENCE LISTING (45 sequences)

SEQ ID NO: 1 - DNA, 3996 bp, Homo sapiens
  cDNA sequence CNTNAP2-001 ENST00000361727 (isoform 1) (1)..(3996)
atgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtgtggaaccctatacggtgcctgtctttttcaacgctacaagttacctggaggtgcccggacggcttaaccaggacctgttctcagtcagtttccagtttaggacatggaaccccaatggtctcctggtcttcagtcactttgcggataatttgggcaatgtggagattgacctcactgaaagcaaagtgggtgttcacatcaacatcacacagaccaagatgagccaaatcgatatttcctcaggttctgggttgaatgatggacagtggcacgaggttcgcttcctagccaaggaaaattttgctattctcaccatcgatggagatgaagcatcagcagttcgaactaatagtccccttcaagttaaaactggcgagaagtacttttttggaggttttctgaaccagatgaataactcaagtcactctgtccttcagccttcattccaaggatgcatgcagctcattcaagtggacgatcaacttgtaaatttatacgaagtggcacaaaggaagccgggaagtttcgcgaatgtcagcattgacatgtgtgcgatcatagacagatgtgtgcccaatcactgtgagcatggtggaaagtgctcgcaaacatgggacagcttcaaatgcacttgtgatgagacaggatacagtggggccacctgccacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 2 - protein, 1331 aa, Homo sapiens
  CASPR2 (1)..(1331)
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCVEPYTVPVFFNATSYLEVPGRLNQDLFSVSFQFRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMCAIIDRCVPNHCEHGGKCSQTWDSFKCTCDETGYSGATCHNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 3 - protein, 27 aa, Artificial Sequence
  Signal peptide
MQAAPRAGCGAALLLWIVSSCLCRAWT

SEQ ID NO: 4 - protein, 1235 aa, Artificial Sequence
  Extracellular Domain
APSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCVEPYTVPVFFNATSYLEVPGRLNQDLFSVSFQFRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMCAIIDRCVPNHCEHGGKCSQTWDSFKCTCDETGYSGATCHNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSA

SEQ ID NO: 5 - protein, 21 aa, Artificial Sequence
  Transmembrane Domain
IIGGVIAVVIFTILCTLVFLI

SEQ ID NO: 6 - protein, 48 aa, Artificial Sequence
  Cytoplasmic Domain
RYMFRHKGTYHTNEAKGAESAESADAAIMNNDPNFTETIDESKKEWLI

SEQ ID NO: 7 - protein, 147 aa, Artificial Sequence
  F5/8 type domain
CDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGC

SEQ ID NO: 8 - protein, 153 aa, Artificial Sequence
  Laminin G-like 1
FKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSC

SEQ ID NO: 9 - protein, 152 aa, Artificial Sequence
  Laminin G-like 2
FRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMC

SEQ ID NO: 10 - protein, 38 aa, Artificial Sequence
  EGF-like 1
IIDRCVPNHCEHGGKCSQTWDSFKCTCDETGYSGATCH

SEQ ID NO: 11 - protein, 207 aa, Artificial Sequence
  Fibrinogen C-terminal
NSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYW

SEQ ID NO: 12 - protein, 165 aa, Artificial Sequence
  Laminin G-like 3
NAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGC

SEQ ID NO: 13 - protein, 40 aa, Artificial Sequence
  EGF-like 2
CSGHCTSYGTNCENGGKCLERYHGYSCDCSNTAYDGTFCN

SEQ ID NO: 14 - protein, 160 aa, Artificial Sequence
  Laminin G-like 4
FSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNC

SEQ ID NO: 15 - DNA, 2124 bp, Artificial Sequence
  delta-FibC-Lam4
atgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtgtggaaccctatacggtgcctgtctttttcaacgctacaagttacctggaggtgcccggacggcttaaccaggacctgttctcagtcagtttccagtttaggacatggaaccccaatggtctcctggtcttcagtcactttgcggataatttgggcaatgtggagattgacctcactgaaagcaaagtgggtgttcacatcaacatcacacagaccaagatgagccaaatcgatatttcctcaggttctgggttgaatgatggacagtggcacgaggttcgcttcctagccaaggaaaattttgctattctcaccatcgatggagatgaagcatcagcagttcgaactaatagtccccttcaagttaaaactggcgagaagtacttttttggaggttttctgaaccagatgaataactcaagtcactctgtccttcagccttcattccaaggatgcatgcagctcattcaagtggacgatcaacttgtaaatttatacgaagtggcacaaaggaagccgggaagtttcgcgaatgtcagcattgacatgtgtgcgatcatagacagatgtgtgcccaatcactgtgagcatggtggaaagtgctcgcaaacatgggacagcttcaaatgcacttgtgatgagacaggatacagtggggccacctgccacgcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 16 - protein, 707 aa, Artificial Sequence
  delta-FibC-Lam4
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCVEPYTVPVFFNATSYLEVPGRLNQDLFSVSFQFRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMCAIIDRCVPNHCEHGGKCSQTWDSFKCTCDETGYSGATCHASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 17 - DNA, 2997 bp, Artificial Sequence
  delta-Disc-Lam1
atgcaggcggctccgcgcgccggctgcggggcagcgctcctgctgtggattgtcagcagctgcctctgcagagcctggacggctccctccacgtcccaaaaatgtgtggaaccctatacggtgcctgtctttttcaacgctacaagttacctggaggtgcccggacggcttaaccaggacctgttctcagtcagtttccagtttaggacatggaaccccaatggtctcctggtcttcagtcactttgcggataatttgggcaatgtggagattgacctcactgaaagcaaagtgggtgttcacatcaacatcacacagaccaagatgagccaaatcgatatttcctcaggttctgggttgaatgatggacagtggcacgaggttcgcttcctagccaaggaaaattttgctattctcaccatcgatggagatgaagcatcagcagttcgaactaatagtccccttcaagttaaaactggcgagaagtacttttttggaggttttctgaaccagatgaataactcaagtcactctgtccttcagccttcattccaaggatgcatgcagctcattcaagtggacgatcaacttgtaaatttatacgaagtggcacaaaggaagccgggaagtttcgcgaatgtcagcattgacatgtgtgcgatcatagacagatgtgtgcccaatcactgtgagcatggtggaaagtgctcgcaaacatgggacagcttcaaatgcacttgtgatgagacaggatacagtggggccacctgccacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 18 - protein, 998 aa, Artificial Sequence
  delta-Disc-Lam1
MQAAPRAGCGAALLLWIVSSCLCRAWTAPSTSQKCVEPYTVPVFFNATSYLEVPGRLNQDLFSVSFQFRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMCAIIDRCVPNHCEHGGKCSQTWDSFKCTCDETGYSGATCHNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 19 - DNA, 2442 bp, Artificial Sequence
  delta-Disc-Lam2
atgcaggcggctccgcgcgccggctgcggggcagcgctcctgctgtggattgtcagcagctgcctctgcagagcctggacggctccctccacgtcccaaaaagcgatcatagacagatgtgtgcccaatcactgtgagcatggtggaaagtgctcgcaaacatgggacagcttcaaatgcacttgtgatgagacaggatacagtggggccacctgccacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 20 - protein, 813 aa, Artificial Sequence
  delta-Disc-Lam2
MQAAPRAGCGAALLLWIVSSCLCRAWTAPSTSQKAIIDRCVPNHCEHGGKCSQTWDSFKCTCDETGYSGATCHNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 21 - DNA, 141 bp, Artificial Sequence
  AAV2 ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 22 - DNA, 141 bp, Artificial Sequence
  AAV2 ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 658 bp, Artificial Sequence
  CAG promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 24 - DNA, 2127 bp, Artificial Sequence
  CASPR2-AB
atgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtgtggaaccctatacggtgcctgtctttttcaacgctacaagttacctggaggtgcccggacggcttaaccaggacctgttctcagtcagtttccagtttaggacatggaaccccaatggtctcctggtcttcagtcactttgcggataatttgggcaatgtggagattgacctcactgaaagcaaagtgggtgttcacatcaacatcacacagaccaagatgagccaaatcgatatttcctcaggttctgggttgaatgatggacagtggcacgaggttcgcttcctagccaaggaaaattttgctattctcaccatcgatggagatgaagcatcagcagttcgaactaatagtccccttcaagttaaaactggcgagaagtacttttttggaggttttctgaaccagatgaataactcaagtcactctgtccttcagccttcattccaaggatgcatgcagctcattcaagtggacgatcaacttgtaaatttatacgaagtggcacaaaggaagccgggaagtttcgcgaatgtcagcattgacatgtgtgcgatcatagacagatgtgtgcccaatcactgtgagcatggtggaaagtgctcgcaaacatgggacagcttcaaatgcacttgtgatgagacaggatacagtggggccacctgccacggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 25 - protein, 708 aa, Artificial Sequence
  CASPR2-AB
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCVEPYTVPVFFNATSYLEVPGRLNQDLFSVSFQFRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMCAIIDRCVPNHCEHGGKCSQTWDSFKCTCDETGYSGATCHGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 26 - DNA, 3327 bp, Artificial Sequence
  CASPR2-CD
atgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 27 - protein, 1108 aa, Artificial Sequence
  CASPR2-CD
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 28 - DNA, 2868 bp, Artificial Sequence
  CASPR2-EF
atgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 29 - protein, 955 aa, Artificial Sequence
  CASPR2-EF
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 30 - DNA, 2790 bp, Artificial Sequence
  CASPR2-GH
atgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtgtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 31 - protein, 929 aa, Artificial Sequence
  CASPR2-GH
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 32 - DNA, 2439 bp, Artificial Sequence
  CASPR2-IJ
atgcaggcggctccgcgcgccggctgcggggcagcgctcctgctgtggattgtcagcagctgcctctgcagagcctggacggctccctccacgtcccaaaaaatcatagacagatgtgtgcccaatcactgtgagcatggtggaaagtgctcgcaaacatgggacagcttcaaatgcacttgtgatgagacaggatacagtggggccacctgccacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttga

SEQ ID NO: 33 - protein, 812 aa, Artificial Sequence
  CASPR2-IJ
MQAAPRAGCGAALLLWIVSSCLCRAWTAPSTSQKIIDRCVPNHCEHGGKCSQTWDSFKCTCDETGYSGATCHNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 34 - DNA, 39 bp, Artificial Sequence
  Primer F1 Universal
ctaccggactcagatctcgagatgcaggcggctccgcgc

SEQ ID NO: 35 - DNA, 42 bp, Artificial Sequence
  Primer F2 Universal
tggcgaccggtggatcccgggaaatgagccattcctttttgc

SEQ ID NO: 36 - DNA, 34 bp, Artificial Sequence
  Primer dFibC-lam4 R1 (AB)
gcgaggccccgtggcaggtggccccactgtatcc

SEQ ID NO: 37 - DNA, 28 bp, Artificial Sequence
  Primer dFibC-lam4 F2 (AB)
cacctgccacggggcctcgccgctgacc

SEQ ID NO: 38 - DNA, 34 bp, Artificial Sequence
  Primer dlam2-EGF1 R1 (CD)
agatagagttacaagaaaagctcaaatttcccac

SEQ ID NO: 39 - DNA, 31 bp, Artificial Sequence
  Primer dlam2-EGF1 F2 (CD)
cttttcttgtaactctatctacgagccttcc

SEQ ID NO: 40 - DNA, 34 bp, Artificial Sequence
  Primer dlam1-EGF1 R1 (EF)
agatagagttgttcaaggcaatgacatctttcag

SEQ ID NO: 41 - DNA, 31 bp, Artificial Sequence
  Primer dlam2-EGF1 F2 (EF)
tgccttgaacaactctatctacgagccttcc

SEQ ID NO: 42 - DNA, 34 bp, Artificial Sequence
  Primer dlam2-EGF1 R1 (GH)
ctccaaccacacaagaaaagctcaaatttcccac

SEQ ID NO: 43 - DNA, 30 bp, Artificial Sequence
  Primer dlam2-EGF1 F2 (GH)
cttttcttgtgtggttggagatactgaccg

SEQ ID NO: 44 - DNA, 31 bp, Artificial Sequence
  Primer Link dDisc-lam2 R1 (IJ)
tgtctatgattttttgggacgtggagggagc

SEQ ID NO: 45 - DNA, 31 bp, Artificial Sequence
  Primer Link dDisc-lam2 F2 (IJ)
gtcccaaaaaatcatagacagatgtgtgccc